(19) United States
(12) Patent Application Publication
(10) Pub. No.: US 2020/0029913 A1
(43) Pub. Date: Jan. 30, 2020

(54) PHYSIOLOGICAL INFORMATION MEASUREMENT DEVICE AND METHOD FOR OUTPUTTING DATA FOR DISPLAYING PHYSIOLOGICAL INFORMATION

(71) Applicant: NIHON KOHDEN CORPORATION, Tokyo (JP)

(72) Inventors: Yoshinori UEDA, Tokorozawa-shi (JP); Kazumasa ITO, Tokorozawa-shi (JP); Hideki FUJISAKI, Tokorozawa-shi (JP)

(73) Assignee: NIHON KOHDEN CORPORATION, Tokyo (JP)

(21) Appl. No.: 16/515,553

(22) Filed: Jul. 18, 2019

(30) Foreign Application Priority Data
Jul. 25, 2018 (JP) 2018-139512

(51) Int. Cl. A61B 5/00; A61B 5/1455

(52) U.S. Cl. CPC A61B 5/7225; A61B 5/742; A61B 5/14551

(57) ABSTRACT

A physiological information measurement device includes one or more processor configured to calculate a first time-averaged value of physiological information of a subject based for a first time period and calculate a second time-averaged value of the physiological information for a second time period that is different from the first time period, and a display configured to display a first graph curve indicating a temporal change of the first time-averaged value and a second graph curve indicating a temporal change of the second time-averaged value.

### CROSS REFERENCE TO RELATED APPLICATION

This application is based on Japanese Patent Application No. 2018-139512 filed on Jul. 25, 2018, the contents of which are incorporated herein by reference.

### BACKGROUND

The presently disclosed subject matter relates to a device for displaying physiological information of a subject and a method for outputting data for displaying the physiological information.

JP-A-2015-107229 discloses a device for displaying physiological information. In this device, various sets of physiological information of a subject are displayed as numerical values. Usually, such a displayed numerical value corresponds to a result which is obtained by performing a time-averaging process on time-varying values of physiological information for a predetermined time period.

The amount of a temporal change of the values of physiological information may be used as an index indicating the instability of physiological information (i.e., the condition of the subject). However, the amount of a temporal change of physiological information (the level of variation from the displayed value) may not be recognized from the displayed value itself which is obtained through a time-averaging process.

### SUMMARY

Accordingly, the presently disclosed subject matter is to assist a user to accurately recognize the condition of the subject.

According to a first aspect of the presently disclosed subject matter, there is provided a physiological information measurement device including:
one or more processors configured to calculate a first time-averaged value of physiological information of a subject for a first time period and calculate a second time-averaged value of the physiological information for a second time period that is different from the first time period; and
a display configured to display a first graph curve indicating a temporal change of the first time-averaged value and a second graph curve indicating a temporal change of the second time-averaged value.

According to a second aspect of the presently disclosed subject matter, there is provided a method for outputting data for displaying physiological information, the method including:
acquiring physiological information of a subject;
calculating a first time-averaged value of the physiological information for a first time period;
calculating a second time-averaged value of the physiological information for a second time period that is different from the first time period; and
outputting data for displaying a first graph curve indicating a temporal change of the first time-averaged value and a second graph curve indicating a temporal change of the second time-averaged value.

According to the above-described configuration, the user can check, through the display, time changes of a plurality of time-averaged values which are calculated from the same physiological information that is acquired from the subject, for respective different time periods. A graph curve indicating a temporal change of a time-averaged value which is calculated for a shorter time period reflects more strongly the variation of acquired physiological information. Therefore, when the consistency between the first and second graph curves is low, it is indicated that the physiological information of the subject is unstable. That is, the user can recognize, through the display, not only the time-averaged value of the physiological information of the subject, but also the stability of the physiological information. Therefore, it is possible to assist the user to accurately recognize the condition of the subject.

### BRIEF DESCRIPTION OF DRAWINGS

FIG. 1 illustrates the functional configuration of a pulse oximeter according to an exemplary embodiment.

FIG. 2 illustrates the procedure of a process which is performed by the pulse oximeter of FIG. 1.

FIG. 3 illustrates a display example of a display of the pulse oximeter of FIG. 1.

FIG. 4 illustrates another display example of the display of the pulse oximeter of FIG. 1.

### DESCRIPTION OF EMBODIMENTS

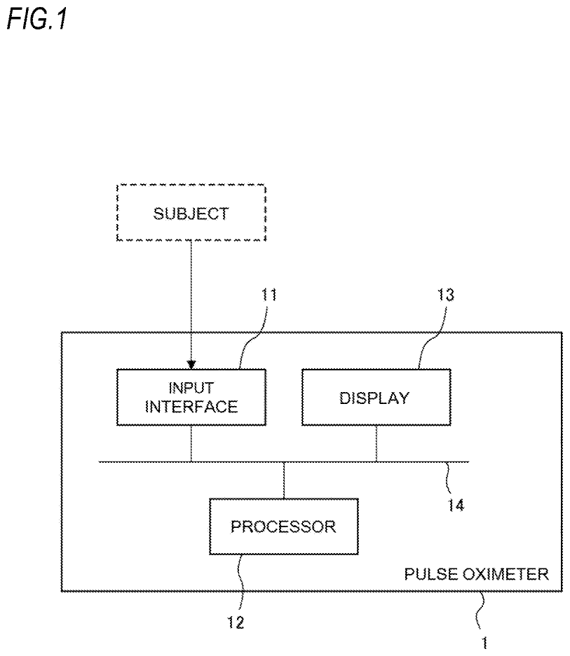

Hereinafter, an exemplary embodiment will be described in detail with reference to the accompanying drawings. FIG. 1 illustrates the functional configuration of a pulse oximeter 1 according to an exemplary embodiment.

The pulse oximeter 1 may include an input interface 11, one or more processors 12, a display 13, and a bus 14. The bus 14 interconnects the input interface 11, the processor 12, and the display 13 such that signals and data can be exchanged between these components.

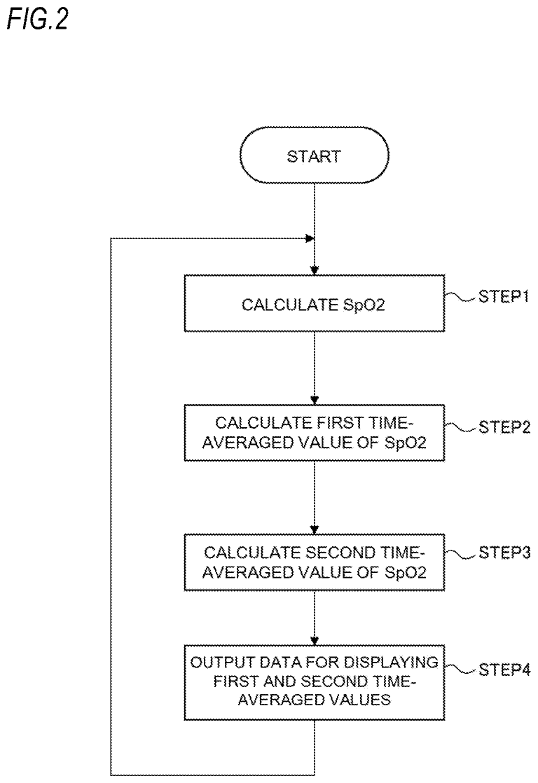

A probe (not illustrated) is attached to the fingertip or earlobe of a subject. The probe may include a light emitter and a light detector. The light emitter emits a red light beam and an infrared light beam. The light detector outputs a signal corresponding to the amounts of the red and infrared light beams which are transmitted through or reflected from a portion where the probe is attached. The signal is supplied to the input interface 11. The input interface 11 may include an appropriate circuit configuration that can convert the supplied signals to data on which the processor 12 can execute a process illustrated in FIG. 2.

The processor 12 is configured to calculate at least the transcutaneous arterial oxygen saturation (SpO2) of the subject based on the data which are supplied through the input interface 11 (STEP 1). The method for calculating the SpO2 is known, and therefore, its detailed description will be omitted. The SpO2 is an example of the physiological information, and the calculation of the SpO2 is an example of acquiring of the physiological information. The calculation of the SpO2 is performed every predetermined time period. For example, the predetermined time period is one second.

Every time when the SpO2 is calculated, the processor 12 calculates a first time-averaged value A1 of the SpO2 for a first time period T1 (STEP 2). Specifically, the average value of a plurality of the SpO2 values which are calculated during a time period of the first time period T1 before the present time is calculated. For example, the first time period T1 is 10 seconds. When the time-varying SpO2 is expressed as f(t), the first time-averaged value A1(t0) at the present time t0 can be indicated by the following expression.

$$A 1 ( to ) = \frac{1}{T 1} {\int }_{to - T 1}^{t 0} f ( t ) dt$$

Every time when the SpO2 is calculated, the processor 12 further calculates a second time-averaged value A2 of the SpO2 for a second time period T2 (STEP 3). Specifically, the average value of a plurality of the SpO2 values which are calculated during a time period of the second time period T2 before the present time is calculated. The second time period T2 is different from the first time period T1. For example, the second time period T2 is three seconds. When the time-varying SpO2 is expressed as f(t), the second time-averaged value A2(t0) at the present time t0 can be indicated by the following expression.

$$A 2 ( to ) = \frac{1}{T 2} {\int }_{to - T 2}^{t 0} f ( t ) dt$$

STEP 2 and STEP 3 may be conducted in the reverse order or at the same time.

Then, the processor 12 outputs data for displaying the first and second time-averaged values A1, A2 of the SpO2 on the display 13. The display 13 displays the first and second time-averaged values A1, A2 based on the data supplied from the processor 12 (STEP 4).

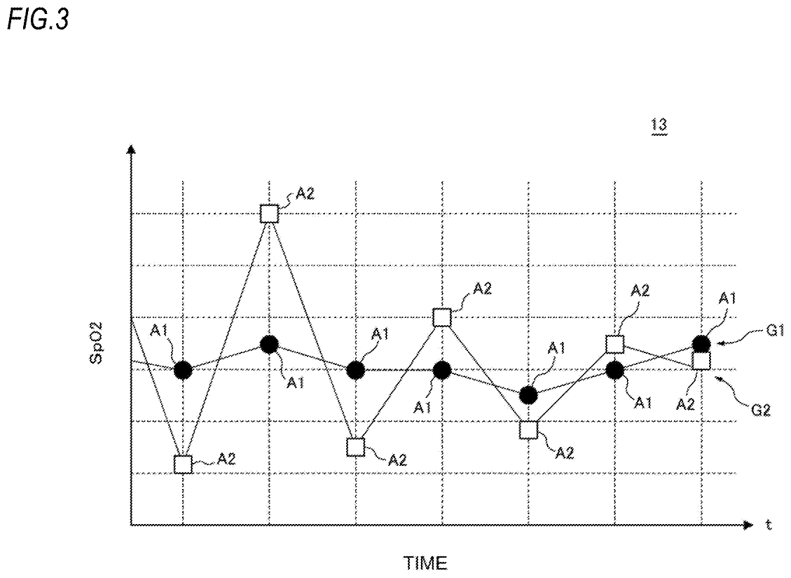

For example, each of the first and second time-averaged values A1, A2 is indicated by a point in the graph curve illustrated in FIG. 3. In FIG. 3, the first time-averaged value A1 is indicated by black circles, and the second time-averaged value A2 is indicated by white squares.

Then, the process returns to STEP 1, and the above-described process is periodically repeated. Accordingly, as illustrated in FIG. 3, a first graph curve G1 indicating a temporal change of the first time-averaged value A1 and a second graph curve G2 indicating a temporal change of the second time-averaged value A2 are displayed on the display 13. At least the latest first time-averaged value A1 may be displayed also as a numerical value.

According to the above-described configuration, the user can check, through the display 13, time changes of a plurality of time-averaged values which are calculated from the SpO2 that is acquired from the same subject, for respective different time periods. The second graph curve G2 indicating a temporal change of the second time-averaged value A2 which is calculated for the shorter second time period T2 reflects more strongly the variation of the acquired SpO2. Therefore, when the consistency between the first graph curve G1 and the second graph curve G2 is low, it is indicated that the SpO2 of the subject is unstable. In the example illustrated in FIG. 3, for example, the consistency between the first graph curve G1 and the second graph curve G2 becomes higher as time passes. Therefore, it is recognized that the SpO2 of the subject is gradually stabilized.

That is, the user can recognize not only the time-averaged value of the SpO2 of the subject, but also the stability of the SpO2, through the display 13. Therefore, it is possible to assist the user to accurately recognize the condition of the subject.

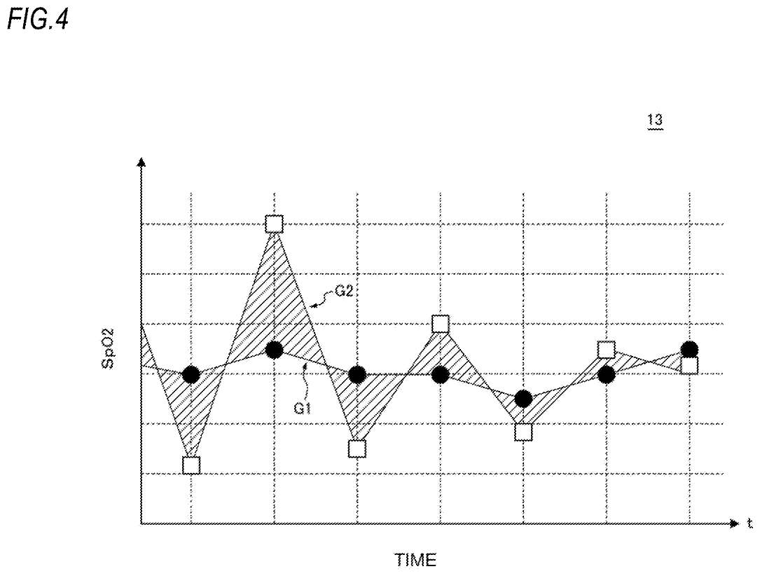

As illustrated in FIG. 4, the one or more regions which is located between the first graph curve G1 and the second graph curve G2 may be displayed in a color that is different from the background color of the display 13.

According to the above-described configuration, it is recognized that the larger the areas of the regions which are displayed in the different color, the higher the inconsistency between the first graph curve G1 and the second graph curve G2, and the lower the stability of the SpO2 value. Therefore, a change in condition of the subject can be recognized more intuitively.

The above-described function of the processor 12 may be realized by a general-purpose microprocessor which operates in cooperation with one or more memory, or by a dedicated integrated circuit such as a microcontroller, an FPGA (Field Programmable Gate Array) or an ASIC (Application Specific Integrated Circuit).

The above-described exemplary embodiment is a mere example for facilitating understanding of the presently disclosed subject matter. The configuration of the exemplary embodiment may be appropriately changed or improved without departing from the spirit of the presently disclosed subject matter.

In the above-described exemplary embodiment, each of the first time-averaged value A1 and the second time-averaged value A2 is calculated as a simple average value. However, at least one of the first time-averaged value A1 and the second time-averaged value A2 may be acquired as the intermediate value or mode value of a plurality of SpO2 values which are acquired during a corresponding time period before the present time.

In the above-described embodiment, the first and second graph curves are displayed on the display. However, the first and second graph curves may be output (printed) on a sheet. Alternatively, data for displaying the first and second graph curves may be output to an external device such as a smartphone. According to this configuration, the first and second graph curves can be checked on a display of the external device.

The configuration which has been described above with reference to the exemplary embodiment can be applied to arbitrary physiological information which can be acquired from a subject and which involves time changes.

1. A physiological information measurement device comprising:
one or more processors configured to calculate a first time-averaged value of physiological information of a subject based for a first time period and calculate a second time-averaged value of the physiological information for a second time period that is different from the first time period; and
a display configured to display a first graph curve indicating a temporal change of the first time-averaged value and a second graph curve indicating a temporal change of the second time-averaged value.

2. The physiological information measurement device according to claim 1,
wherein a region which is located between the first graph curve and the second graph curve is displayed by a color that is different from a background color of the display.

3. A method for outputting data for displaying physiological information, the method comprising:
acquiring physiological information of a subject;
calculating a first time-averaged value of the physiological information for a first time period;
calculating a second time-averaged value of the physiological information for a second time period that is different from the first time period; and
outputting data for displaying a first graph curve indicating a temporal change of the first time-averaged value and a second graph curve indicating a temporal change of the second time-averaged value.
